(19) United States
(12) Patent Application Publication
(10) Pub. No.: US 2023/0263627 A1
(43) Pub. Date: Aug. 24, 2023

(54) GROWTH-ACCOMMODATING VALVE SYSTEM

(71) Applicant: THE REGENTS OF THE UNIVERSITY OF CALIFORNIA, Oakland, CA (US)

(72) Inventors: Arash Kheradvar, Irvine, CA (US); Daryl C. Nguyen, Irvine, CA (US)

(21) Appl. No.: 18/308,239

(22) Filed: Apr. 27, 2023

Related U.S. Application Data
(63) Continuation-in-part of application No. 17/088,989, filed on Nov. 4, 2020.
(60) Provisional application No. 63/335,455, filed on Apr. 27, 2022.
(60) Provisional application No. 62/932,735, filed on Nov. 8, 2019.

(51) Int. Cl. A61F 2/24

(52) U.S. Cl. CPC A61F 2/2418; A61F 2/2433; A61F 2210/0057

(57) ABSTRACT

A growth-accommodating prosthetic valve system that can accommodate a patient's growth over time. The system features an expandable stent that can expand from a contracted configuration to an expanded configuration and leaflets attached to the scaffold that is sewn along a path such that the length of the path stays generally the same as the scaffold moves from the contracted configuration to the expanded configuration.

### CROSS-REFERENCES TO RELATED APPLICATIONS

This application is a non-provisional and claims benefit of U.S. Provisional Application No. 63/335,455 filed Apr. 27, 2022, the specification of which is incorporated herein in their entirety by reference.

This application is a continuation-in-part and claims benefit of U.S. patent application Ser. No. 17/088,989 filed Nov. 4, 2020, which is a non-provisional and claims benefit of U.S. Provisional Application No. 62/932,735 filed Nov. 8, 2019, the specifications of which are incorporated herein in their entirety by reference.

### STATEMENT REGARDING FEDERALLY SPONSORED RESEARCH OR DEVELOPMENT

This invention was made with Government support under Grant No. 1R21HD105889-01, awarded by the National Institutes of Health. The Government has certain rights in the invention.

### BACKGROUND OF THE INVENTION

### Field of the Invention

The present invention relates to prosthetic valve systems, namely, a valve system that accommodates a child's growth through balloon expansion and methods of use. A non-limiting application of such a valve system is to replace a diseased pulmonary valve, which is commonly involved in Congenital Heart Disease (CHD).

### Background Art

Congenital heart defects (CHDs) occur in −1% of births in the U.S. and Europe. Presently, it is estimated that at least 1 million children are living with CHD in the U.S. Owing to improved medical and surgical care, it is estimated that 83% of babies born with CHD in the U.S. survive infancy. In some of these patients, it becomes necessary to replace a heart valve while they are still at a growing age. In those cases, if the implanted heart valve does not accommodate for the growth of the child, it would need to be replaced multiple times before the child is fully grown into an adult.

### BRIEF SUMMARY OF THE INVENTION

The present invention provides a growth-accommodating prosthetic valve system that is capable of expanding to accommodate the growth of a child. The valve system of the present invention may accommodate growth through a balloon expansion and can be implanted through transcatheter means.

Briefly, for example, the present invention features a growth-accommodating prosthetic valve system comprising an expandable stent, e.g., a radially expandable stent, and a plurality of leaflets each sutured to the expandable stent along a path, wherein the stent and leaflets together are configured to form a valve. The path of attachment has a length, and the length stays the same (or generally the same) as the stent expands (e.g., radially) from at least a first position (e.g., contracted configuration) to a second position (e.g., expanded configuration). The expandable stent comprises a plurality of support beams interconnected by joints that together form a web-like cylindrical cage, and each leaflet is sutured to at least one support beam. The path may be defined as the length of the support beam or combination of support beams (and joints) to which a leaflet is sutured. As the expandable stent, the height of the expandable stent decreases to accommodate the expansion of the diameter of the stent. The diameter of the expandable stent expands while keeping its full coaptation of leaflets.

In some embodiments, the present invention features a prosthetic valve system that accommodates growth, said system comprises a) an expandable stent; and b) a plurality of leaflets, each attached to the expandable stent along a path so as to form a valve, wherein the path has a length that stays the essentially the same as the stent expands from at least a first position to a second position. As the expandable stent expands between the first position and the second position, the angle of the path that attaches the leaflets to the expandable stent changes with respect to the vertical axis of the expandable stent to accommodate an increased diameter of the expandable stent.

In some embodiments, the present invention features a prosthetic valve system that accommodates growth of a patient, said system comprises a) an expandable stent; and b) a plurality of leaflets, each attached to the expandable stent along a path so as to form a valve, wherein the path has a length that stays the essentially the same as the stent expands from at least a first position to a second position. As the expandable stent radially expands, the height of the expandable stent (decreases to accommodate expansion of the diameter of the expandable stent.

In some embodiments, the lower limit of the diameter of the expandable stent is 9 mm. In some embodiments, the upper limit of the diameter of the expandable stent is 25 mm.

In some embodiments, the leaflets are constructed from a material comprising biological tissue. In some embodiments, the leaflets are constructed from a material comprising a stretchable polymeric material.

In some embodiments, the system further comprises a skirt around its outer surface to mitigate paravalvular leak. In some embodiments, the skirt is constructed from a material comprising a stretchable polymer.

One of the unique and inventive technical features of the present invention is the use of excessive leaflet tissue for the system in the contracted configuration that allows for the expansion of the system to an expanded configuration (or a configuration between the contracted and expanded configuration, e.g., expanding from a first diameter of 12 mm to a second diameter of 20 mm, to accommodate the child's growth, and also does not require more blood pressure to open the valve despite the excessive tissue. Further, the present invention features an attachment path for the leaflets that does not change (or generally does not change) so as to avoid tearing of the leaflet during expansion. The configuration of the leaflets with the stent is important so that as the stent expands, the leaflets still provide valve closure but at different three-dimensional configurations (e.g., see FIG. 2A, FIG. 2B).

Embodiments of the present invention can be freely combined with each other if they are not mutually exclusive.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows an embodiment of a growth-accommodating stented valve system of the present invention in a contracted configuration.

FIG. 1B shows the embodiment of a growth-accommodating stented valve system of FIG. 1A in an expanded configuration.

FIG. 2A shows the closure of the leaflets for the embodiment in FIG. 1A.

FIG. 2B shows the closure of the leaflets for the embodiment in FIG. 1B.

FIG. 3A shows the path (see bold line) of the attachment of a leaflet to the support beams and joints. The system is in a contracted configuration.

FIG. 3B shows the path (see bold line) of the attachment of a leaflet to the support beams and joints. The system is in an expanded configuration.

FIG. 4A shows the length of the path (see bold line) of a leaflet. The system is in a contracted configuration.

FIG. 4B shows the length of the path (see bold line) of a leaflet. The system is in an expanded configuration.

### DETAILED DESCRIPTION OF THE INVENTION

### Definitions

Unless otherwise defined herein, scientific and technical terms used in connection with the present application shall have the meanings that are commonly understood by those of ordinary skill in the art. Further, unless otherwise required by context, singular terms shall include pluralities, and plural terms shall include the singular. Thus, as used in this specification and the appended claims, the singular forms “a,” “an,” and “the” include plural referents unless the context clearly indicates otherwise.

One skilled in the art would recognize that the term “heart” refers to a muscular organ located just behind and slightly left of the breastbone that pumps blood through the network of arteries and veins called the cardiovascular system. The heart has four chambers comprising the right atrium, the right ventricle, the left atrium, and the left ventricle. Additionally, the heart has four valves comprising the mitral valve (located between the atria), the tricuspid valve (located between the ventricles), the aortic valve (located between the left ventricle from the aorta), and the pulmonary valve (located between the right ventricle and pulmonary artery).

Each valve in the heart is made up of strong, thin flaps of tissue called leaflets or cusps. As used herein, “cusps” or “leaflets” may be used interchangeably. A leaflet may refer to one of the triangular segments of a valve in the heart, which opens and closes with the flow of blood. Leaflets open to let blood move forward through the heart during half of the heartbeat. They close to keep blood from flowing backward during the other half of the heartbeat.

As used herein, “coapting” refers to one or more leaflets fitting together closely to prevent backward blood flow.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject/patient is a human. In one embodiment, the subject is a mammal (e.g., a human) having a disease, disorder, or condition described herein. In another embodiment, the subject is a mammal (e.g., a human) at risk of developing a disease, disorder, or condition described herein.

The present invention features a growth-accommodating prosthetic valve system (100) that is capable of expanding to accommodate the growth of a child.

The present invention features a prosthetic valve system (100). In some embodiments, the system (100) comprises a) an expandable stent (110), and b) a plurality of leaflets (210) each attached to the expandable stent (110) along a path (220) so as to form a heart valve. In some embodiments, the path (220) has a length that stays the same as the stent (110) expands from a first position (201) to a second position (202). The valve system (100) described herein is twice expandable and may be deployed into a first position (201), e.g., in the heart, and then expanded into a second position (202), e.g., to accommodate heart growth.

In some embodiments, the valve system (100) described herein is twice expandable and may be deployed into a first position (201) in the heart and in a future date may be expanded into a second position (202) to accommodate heart growth.

The present invention features a prosthetic valve system (100) that accommodates growth, said system comprises a) an expandable stent (110); and b) a plurality of leaflets (210), each attached to the expandable stent (110) along a path (220) so as to form a valve, wherein the path (220) has a length that stays the essentially the same as the stent (110) expands from at least a first position (201) to a second position (202). As the expandable stent (110) expands between the first position (201) and the second position (202), the angle of the path (220) that attaches the leaflets (210) to the expandable stent (110) changes with respect to the vertical axis of the expandable stent (110) to accommodate an increased diameter (141) of the expandable stent (110).

The present invention features a prosthetic valve system (100) that accommodates growth of a patient, said system comprises a) an expandable stent (110); and b) a plurality of leaflets (210), each attached to the expandable stent (110) along a path (220) so as to form a valve, wherein the path (220) has a length that stays the essentially the same as the stent (110) expands from at least a first position (201) to a second position (202). As the expandable stent (110) radially expands, a height (142) of the expandable stent (110) decreases to accommodate expansion of the diameter (141) of the expandable stent (110).

In some embodiments, the system (100) comprises a) an expandable stent (110), and b) a plurality of leaflets (210) each attached to the expandable stent (110) along a path (220) so as to form a heart valve. In some embodiments, the path (220) has a length that stays essentially the same as the stent (110) expands from at least a first position (201) to a second position (202). As the expandable stent (110) expands between the first position (201) and the second position (202), the angle of the path (220) that attaches the leaflets (210) to the expandable stent (110) changes with respect to the vertical plane of the expandable stent (110) to accommodate an increased diameter (141) of the expandable stent (110).

In some embodiments, the system (100) comprises a) an expandable stent (110), and b) a plurality of leaflets (210) each attached to the expandable stent (110) along a path (220) so as to form a heart valve. In some embodiments, the path (220) has a length that stays essentially the same as the stent (110) expands from at least a first position (201) to a second position (202). As the expandable stent (110) radially expands, the height (142) of the expandable stent (110) decreases to accommodate expansion of the diameter (141) of the expandable stent (110).

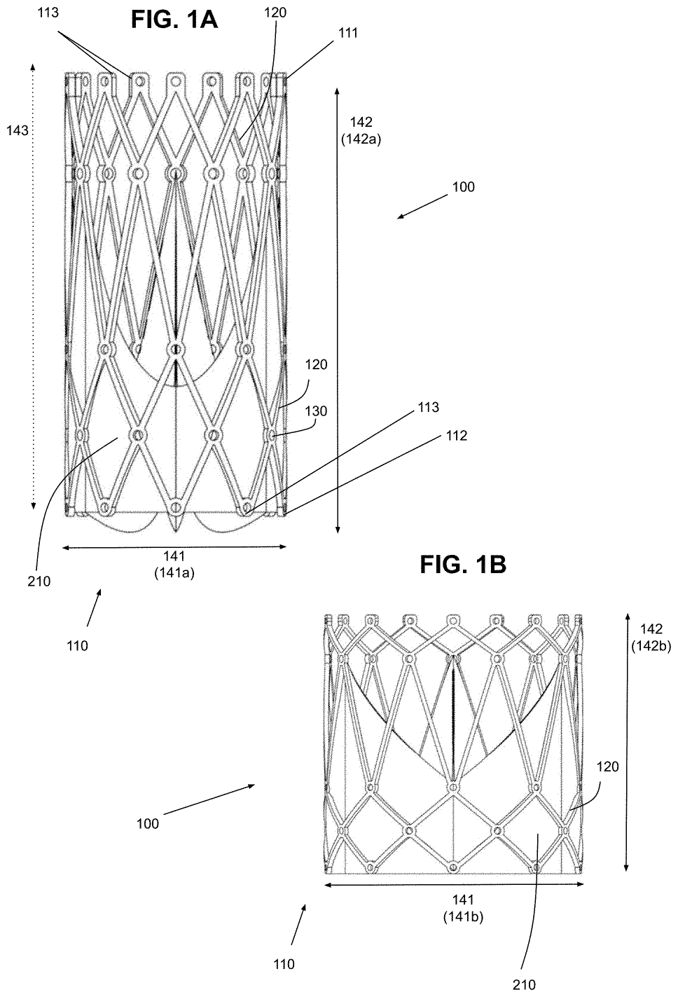

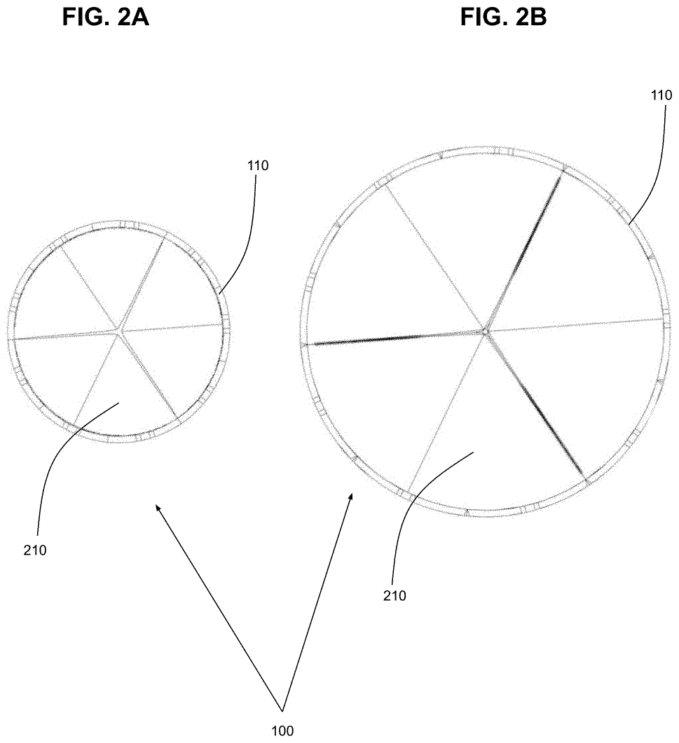

Referring now to FIG. 1A, FIG. 1B, FIG. 2A, and FIG. 2B, the present invention features a growth-accommodating prosthetic valve system (100) comprising an expandable stent (110) and a plurality of leaflets (210) attached to the expandable stent (110) to form a valve. The system (100), e.g., the diameter (141) of the system (100), can expand between or from at least a first position (201), as shown in FIG. 1A with a first diameter (141a) and a first height (142a) to a second position (202), as shown in FIG. 1B with a second diameter (141b) and a second height (142b). The height is measured from the top end (111) to the bottom end (112).

In some embodiments, the first height (142a) and the second height (142b) may be determined based on the inherent properties of the materials used to create the expandable stent.

Before deployment, the valve system (100) described herein may be crimped into a delivery position. In certain embodiments, the delivery position is non-functional, meaning that the valve system (100) cannot function as a valve while in this position. In some embodiments, the delivery position may be utilized prior to deploying the valve system into a first position (201).

In some embodiments, the first position (201) comprises a contracted configuration, e.g., as compared to the second position (202). In other embodiments, the first position (201) comprises a partially expanded configuration, e.g., as compared to the second position (202). In some embodiments, the second position (202) comprises an expanded configuration. In other embodiments, the second position (202) comprises a fully expanded configuration, e.g., as compared to the first position (201).

In some embodiments, the expandable stent (110) may be constructed from a plurality of support beams (120) interconnected by joints (130) that together form a web-like cylindrical cage.

As shown in FIG. 1A and FIG. 1B, the stent (or expandable cage) has a top end (111) and a bottom end (112). In some embodiments, the web-like configuration of the interconnected joints extends along the length of the stent, e.g., from the top end (111) to the bottom end. In some embodiments, the top end comprises a ring of joint elbows spaced a distance apart. In some embodiments, the bottom end is configured with a ring of joint elbows spaced a distance apart.

In some embodiments, the expandable stent, e.g., the support beams and/or joints, may be constructed from a material comprising a chromium-cobalt alloy. In other embodiments, the expandable stent may be constructed from a variety of other materials suitable for a desired biological application. Non-limiting examples of suitable material include, but are not limited to, cobalt chromium, stainless steel, shape-memory materials, polymers, plastic, self-expanding Nitinol, thermal shape memory Nitinol, etc. In other embodiments, the expandable stent, e.g., the support beams and/or joints, may be constructed from a material comprising a magnetic shape memory alloy.

In some embodiments, the valve system (100) described herein is implanted/deployed via a transcatheter means. In some embodiments, the valve system (100) described herein is implanted/deployed via a surgical means.

In some embodiments, the valve system (100) described herein is expandable using a balloon via a transcatheter means. In other embodiments, the valve system (100) described herein is expandable via surgical means.

In some embodiments, the valve system (100) is self-expandable. In other embodiments, the valve system (100) may be expanded with a balloon.

In some embodiments, a first balloon is used to expand the prosthetic valve system (100) into the first position (201), and a second balloon is used to expand the prosthetic valve system (100) into the second position (202). In some embodiments, the prosthetic valve system (100) self-expands into the first position (201), and a second balloon is used to expand the prosthetic valve system (100) into the second position (202).

In some embodiments, the valve is deployed into the first position (201) using a first balloon, and the valve is expanded into the second position (202) using a second inflatable balloon. In another embodiment, the valve is deployed into the first position (201) using a first balloon, and in a future date the valve is expanded into the second position (202) using a second inflatable balloon.

In other embodiments, the expandable stent (110) is expanded into a second position (202) magnetically, e.g., in embodiments where the expandable stent (110) comprises a ferroelectric material, e.g., a magnetic shape memory alloy.

In some embodiments, the expandable stent (110) may also be equipped with at least one bioactive agent for biologically inspired applications (e.g., the expandable stent (110) comprises a drug-eluting stent). Non-limiting examples of the bioactive agent include analgesics/antipyretics, antiasthmatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antipsychotic agents, antimanic agents, antiarrhythmics, antiarthritic agents, antigout agents, anticoagulants, thrombolytic agents, antifibrinolytic agents, platelet aggregation inhibitor agents, and antibacterial agents, antiviral agents, antimicrobials, anti-infective agents, or any combination thereof.

The prosthetic valve system (100) is configured to expand from the first position (201) to the second position (202) such that the diameter of the stent (110) in the second position (201) is greater than the diameter of the first position (201).

The prosthetic valve system is capable of expanding from at least a first position to a second position, wherein in the second position, the diameter of the stent is greater than the diameter of the stent in the first position. In some embodiments, the diameter of the stent in the second position is double (2 times) that of the first position. In some embodiments, the diameter of the stent in the second position is 1.5 times that of the first position. In some embodiments, the diameter of the stent in the second position is 1.6 times that of the first position. In some embodiments, the diameter of the stent in the second position is 1.7 times that of the first position. In some embodiments, the diameter of the stent in the second position is 1.8 times that of the first position. In some embodiments, the diameter of the stent in the second position is 1.9 times that of the first position. In some embodiments, the diameter of the stent in the second position is at least 2 times that of the first position. In some embodiments, the diameter of the stent in the second position is 2.5 times that of the first position.

In some embodiments, the expandable stent (110) has a diameter with a lower limit of greater than 7 mm, e.g., the first diameter (141a) in the first position is greater than 7 mm. In some embodiments, the expandable stent (110) has a diameter with a lower limit of 8 mm, e.g., the first diameter (141a) in the first position is 8 mm. In some embodiments, the expandable stent (110) has a diameter with a lower limit of 9 mm, e.g., the first diameter (141a) in the first position is 9 mm. In some embodiments, the expandable stent (110) has a diameter with a lower limit of 10 mm, e.g., the first diameter (141a) in the first position is 10 mm. In some embodiments, the expandable stent (110) has a diameter with a lower limit of 11 mm, e.g., the first diameter (141a) in the first position is 11 mm. In some embodiments, the expandable stent (110) has a diameter with a lower limit of 12 mm, e.g., the first diameter in the first position is 12 mm. The present invention is not limited to the aforementioned ranges of diameters.

In some embodiments, the expandable stent (110) has a diameter with an upper limit of 20 mm, e.g., the second diameter (141b) in the second position is 20 mm. In some embodiments, the expandable stent (110) has a diameter with an upper limit of 21 mm, e.g., the second diameter (141b) in the second position is 21 mm. In some embodiments, the expandable stent (110) has a diameter with an upper limit of 22 mm, e.g., the second diameter (141b) in the second position is 22 mm. In some embodiments, the expandable stent (110) has a diameter with an upper limit of 23 mm, e.g., the second diameter (141b) in the second position is 23 mm. In some embodiments, the expandable stent (110) has a diameter with an upper limit of 24 mm, e.g., the second diameter (141b) in the second position is 24 mm. In some embodiments, the expandable stent (110) has a diameter with an upper limit of 25 mm, e.g., the second diameter (141b) in the second position is 25 mm. The present invention is not limited to the aforementioned ranges of diameters.

In some embodiments, the system (100) comprises three leaflets (210). In some embodiments, the system (100) comprises two leaflets (210). In some embodiments, the system (100) comprises four leaflets (210).

The leaflets (210) are attached to the expandable stent (110), e.g., to form a valve.

For example, the leaflets (210) may be attached to the expandable stent (110) through sewing, e.g., suturing. In some embodiments, the leaflets (210) are attached via polymeric material. The present invention is not limited to the aforementioned methods or materials for attaching the leaflets (210) to the scaffold (110).

In some embodiments, the leaflets (210) are laser-cut from porcine pericardial tissue, e.g., with a thickness of about 200 microns, and then sewn to the expandable stent or attached via other methods. Porcine pericardial tissue is a standard material because it is less prone to infective endocarditis. However, it is to be understood that the leaflets are not limited to porcine pericardium tissue.

The leaflets (210) may be made from a variety of materials with thicknesses, e.g., varying from about 50-500 microns. The leaflets may be made from a variety of materials with thicknesses, e.g., varying from about 100 to 300 microns. In some embodiments, the leaflets may be made from a variety of materials with thicknesses varying from about 50-100 microns, or about 100-150 microns, or about 150-200 microns, or about 200-250 microns, or about 250-300 microns, or about 350-400 microns, or about 450-500 microns. In some embodiments, the system is manufactured with thin leaflet tissue, such as to keep the profile of the valve system low.

The leaflets may comprise a biological or synthetic material. Non-limiting examples of suitable materials include but are not limited to, natural membranes, polymer material (natural or synthetic), engineered biological tissue, biological valvular leaflet tissue, pericardial tissue or crosslinked pericardial tissue, other non-pericardial tissue or xenogeneic valve tissue. In one embodiment, the tissue may be procured from human, bovine, equine, ovine, or other animals. In another embodiment, the crosslinked pericardial tissue is crosslinked with a crosslinking agent such as formaldehyde, glutaraldehyde, dialdehyde starch, antibiotics, glyceraldehydes, cyanamide, diimides, diisocyanates, dimethyl adipimidate, neomycin, carbodiimide, epoxy compound, or any mixture thereof.

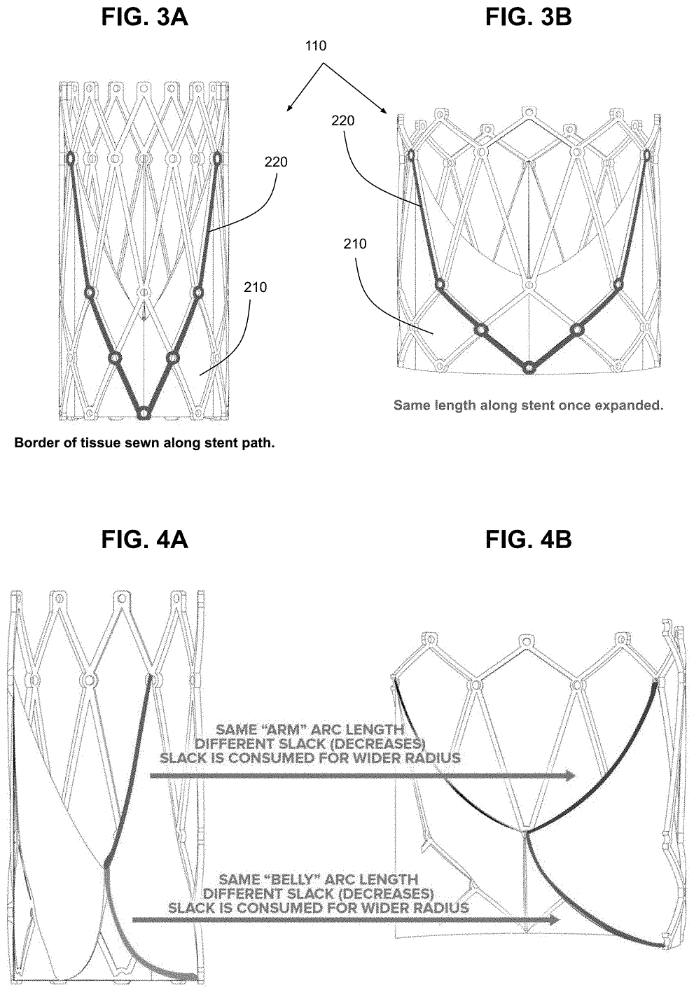

Referring to FIG. 3A, FIG. 3B, FIG. 4A, and FIG. 4B, each leaflet (210) has a path (220), e.g., a side, an edge, etc., that is attached to one or more support beams (120) and/or joints (130). As shown in the figures, each leaflet (210) has a first end that is attached to a first attachment point (a support beam and/or joint). Each leaflet (210) has a second side end attached to a second attachment point (a support beam and/or joint), wherein the first attachment point is a distance apart from the second attachment point. In some embodiments, the path (220) is defined as a length along a support beam (120) or a combination of support beams (120) to which a leaflet (210) is attached.

In some embodiments, each leaflet (210) is attached to the stent along the length of the path (220). In some embodiments, each leaflet (210) is sutured to the stent along the length of the path (220). Each leaflet (210) may be attached to the stent using one or a plurality of sutures. In some embodiments, a single thread is used to suture a leaflet (210) along a path (220).

Each path (220) has a length. The length of the path (220) is the same (or generally the same) whether the system (100) is in the contracted configuration or expanded configuration (or between said configurations).

In some embodiments, the size (e.g., the length) of the leaflets (210) does not change; however, the 3D orientation of the leaflets (210) changes which allows for the expansion of the expandable stent (110), e.g., growth accommodation.

As the system (100) expands between the first position and the second position, the angle of the path (220) that connects the leaflets to the expandable stent changes with respect to the vertical plane (143) of the expandable stent (110) to accommodate an increased diameter (141) of the expandable stent (110). In some embodiments, as the expandable stent (110) expands between the first position (201) and the second position (202), the angle of the path (220) that attaches the leaflets (210) to the expandable stent (110) changes with respect to the vertical plane of the expandable stent (110) to accommodate an increased diameter (141) of the expandable stent (110). In other embodiments, as the expandable stent (110) expands between the first position (201) and the second position (202), the angle of the path (220) that attaches the leaflets (210) to the expandable stent (110) changes with respect to the vertical axis of the expandable stent (110) to accommodate an increased diameter (141) of the expandable stent (110).

As the system (100) expands, the length (142) of the expandable stent (110) decreases to accommodate the expansion of the diameter (141) of the expandable stent (110).

As the expandable stent (110) radially expands, the height (142) of the expandable stent (110) decreases to accommodate expansion of the diameter (141) of the expandable stent (110).

The system is manufactured with thin leaflet tissue, and in the contracted configuration (or between the contracted configuration and expanded configuration) the angle between the leaflet tissue and the stent is acute. This allows for the expansion of the system as the patient grows. When the system is in the expanded configuration (e.g., the second position), the angle between the leaflet tissue and the stent increases (e.g., is obtuse). Thus, the size of the leaflet tissue never changes between the contracted configuration (e.g., the first position (201)) or the expanded configuration (202)), but the 3D orientation of the leaflets (210) changes, e.g., the angle between the leaflet and the stern increases as the valve is expanded from a first position to a second position.

Without wishing to limit the present invention to any theory or mechanism, it is believed that the thin leaflet tissue helps prevent the need for higher blood pressure when the system is in the contracted configuration (e.g., the first position).

Furthermore, the leaflets described herein have the advantage of only altering their 3D orientation and not their size during the expansion of the valve from a contracted to an expanded configuration. As a result, the necessity to fold the leaflets to fit within the contracted configuration is eliminated. In some embodiments, the leaflets are not folded when the valve is in a contracted configuration (e.g., a first position).

In some embodiments, the leaflets (210) are not folded when the expandable stent (110) is in the first position (201; e.g., a contracted configuration). In some embodiments, the leaflets (210) are not folded when the expandable stent (110) is in the second position (202; e.g., an expanded configuration). In some embodiments, the leaflets (210) are not folder when the expandable stent (110) is in the first position (201) and the second position (202).

In some embodiments, the leaflets (210) are not folded when the system (100) is in the first position (201; e.g., a contracted configuration). In some embodiments, the leaflets (210) are not folded when the system (100) is in the second position (202; e.g., an expanded configuration). In some embodiments, the leaflets (210) are not folder when the system (100) is in the first position (201) and the second position (202).

In some embodiments, the diameter (141) of the expandable stent (110) expands while keeping its full coaptation of leaflets (210). In some embodiments, the diameter (141) of the expandable stent (110) expands while its full coaptation of leaflets is kept intact (210).

In some embodiments, the system further comprises a skirt around its outer surface (e.g., the outer surface of the expandable stent (110)) to mitigate paravalvular leak. In some embodiments, the skirt is constructed from a material comprising a stretchable polymer. In some embodiments, the skirt is constructed from a material comprising biological tissue, e.g., pericardial tissue.

The present invention also provides a method of treating or ameliorating congenital heart defects in a patient in need thereof. The method may comprise implanting in said patient a system (100) as described herein to replace a valve.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A prosthetic valve system (100), said system comprising:
a) an expandable stent (110), and
b) a plurality of leaflets (210) each attached to the expandable stent (110) along a path (220) so as to form a heart valve, wherein the path (220) has a length that stays the same as the stent (110) expands from a first position (201) to a second position (202);
wherein the valve system (100) is twice expandable, wherein the valve is deployed into the first position (201) in a heart and then expanded at a future date into the second position (202) to accommodate growths of the heart.

2. The prosthetic valve system (100) of claim 1, wherein the prosthetic valve system (100) is configured to expand from the first position to the second position such that the diameter of the stent in the second position is greater than the diameter of the first position.

3. The prosthetic valve system (100) of claim 1, wherein the valve is deployed into the first position (201) using a first balloon, and in a future date, the valve is expanded into the second position (202) using a second inflatable balloon.

4. The prosthetic valve system (100) of claim 1, wherein the first position (201) comprises a partially expanded configuration and the second position (202) comprises a fully expanded configuration.

5. The prosthetic valve system (100) of claim 1, wherein in the first position (201), the expandable stent (110) has a diameter (141) of from 9 to 12 mm.

6. The prosthetic valve system (100) of claim 1, wherein in the first position (201) the expandable stent (110) has a diameter (141) of about 12 mm.

7. The prosthetic valve system (100) of claim 1, wherein in the second position (202) the expandable stent (110) has a diameter (141) from 20 to 25 mm.

8. The prosthetic valve system (100) of claim 1, wherein in the second position (202) the expandable stent (110) has a diameter of 20 mm.

9. The prosthetic valve system (100) of claim 1, wherein the expandable stent (110) comprises a plurality of support beams (120) interconnected by joints (130) that together form a web-like cylindrical cage.

10. The prosthetic valve system (100) of claim 9, wherein each leaflet (210) is attached to at least one support beam (120), wherein the path (220) is defined as a length along a support beam (120) or a combination of support beams (120) to which a leaflet (210) is attached.

11. The prosthetic valve system of (100) claim 1, wherein as the expandable stent (110) expands between the first position (201) and the second position (202), the angle of the path (220) that attaches the leaflets (210) to the expandable stent (110) changes with respect to the vertical axis of the expandable stent (110) to accommodate an increased diameter (141) of the expandable stent (110).

12. The prosthetic valve system (100) of claim 1, wherein as the expandable stent (110) radially expands, a height (142) of the expandable stent (110) decreases to accommodate expansion of the diameter (141) of the expandable stent (110).

13. The prosthetic valve system (100) of claim 1, wherein the leaflets (210) are constructed from a material comprising biological tissue or wherein the leaflets (210) are constructed from a material comprising a stretchable polymeric material.

14. The prosthetic valve system (100) of claim 1, further comprising a skirt around its outer surface to mitigate paravalvular leak.

15. The prosthetic valve system (100) of claim 14, wherein the skirt is constructed from a material comprising a stretchable polymer, or wherein the skirt is constructed from a material comprising a biological tissue.

16. The prosthetic valve system (100) of claim 1, wherein the diameter (141) of the expandable stent (110) expands while its full coaptation of leaflets (210) is kept intact.

17. The prosthetic valve system (100) of claim 1, wherein each of the plurality of leaflets (210) is sutured to the expandable stent (110) along the path (220).

18. A prosthetic valve system (100) that accommodates growth of a patient, said system comprising:
a) an expandable stent (110); and
b) a plurality of leaflets (210), each attached to the expandable stent (110) along a path (220) so as to form a valve, wherein the path (220) has a length that stays the essentially the same as the stent (110) expands from at least a first position (201) to a second position (202);
wherein as the expandable stent (110) expands between the first position (201) and the second position (202), the angle of the path (220) that attaches the leaflets (210) to the expandable stent (110) changes with respect to the vertical axis of the expandable stent (110) to accommodate an increased diameter (141) of the expandable stent (110).

19. A prosthetic valve system (100) that accommodates growth, said system comprising:
a) an expandable stent (110); and
b) a plurality of leaflets (210), each attached to the expandable stent (110) along a path (220) so as to form a valve, wherein the path (220) has a length that stays the essentially the same as the stent (110) expands from at least a first position (201) to a second position (202);
wherein as the expandable stent (110) radially expands, a height (142) of the expandable stent (110) decreases to accommodate expansion of the diameter (141) of the expandable stent (110).
